(19) United States
(12) Patent Application Publication
(10) Pub. No.: US 2022/0152248 A1
(43) Pub. Date: May 19, 2022

(54) DOOR HANDLE SANITIZER SYSTEM, DEVICE, AND METHOD

(71) Applicant: 99Point9 Hygiene Limited, Chesterfield (GB)

(72) Inventor: Simon Sassoon, Fowey (GB)

(21) Appl. No.: 16/950,321

(22) Filed: Nov. 17, 2020

(51) Int. Cl. A61L 2/22; A61L 2/24

(52) U.S. Cl. CPC A61L 2/22; A61L 2202/14; A61L 2202/15; A61L 2/24

(57) ABSTRACT

A system, device, and method render germ-free door handles in a commercial environment by sanitizing such door handles through the dispensing of a germicide, such as a disinfectant, antibacterial solution, or cleansing agent, onto the door handles at controlled intervals. The controlled intervals are determined by a set of controls which establish a series of settings corresponding to a different amount of time that a valve member is in an operating state.

### FIELD OF THE DISCLOSURE

The present invention relates to a system, device, and method for rendering germ-free (sanitizing) door handles in a commercial environment, and more particularly, to a device for dispensing a germicide, such as a disinfectant, antibacterial solution, or cleansing agent, onto a door handle at controlled intervals and to a valve attachment suitable for use therewith.

### BACKGROUND OF THE DISCLOSURE

Many people are reluctant to use public restrooms due to actual or perceived sanitary conditions of those restrooms. However, in some instances, such use is unavoidable. These people try to avoid touching any surface after they have washed their hands. However, touching a surface of the restroom is nearly unavoidable because sometimes the person must touch the handle of the door to exit the restroom. Some people carry gloves, wipes or the like to use in such public restrooms. Some people take an extra paper towel to use to cover the door handle. All of these techniques work but are burdensome and not efficient.

Some prior art methods suggest covering a handle of a door so a person opening the door can avoid direct contact with the handle. Covering a door handle, while effective in preventing a person from contacting the door handle during operation of the door, does little to clean or disinfect the door handle. Furthermore, the cover then becomes a source of contamination, germs, bacteria and the like. Therefore, there is a need for efficiently and effectively sanitizing the handle of a door. This typically requires a person to carry a liquid spray bottle into a room, spray a disinfectant or antibacterial liquid onto the door handle and then wipe the handle clean. This procedure can be cumbersome and inefficient, requiring a person to carry items with him or her for the cleaning procedure.

Still further, some people, often nicknamed germaphobes, wonder when the last time a door handle was cleaned, and even if there is some form of protection for this person, they are uncomfortable touching the door handle. These people are not satisfied by the mere existence of some means for cleaning or sanitizing the door handle, rather, they might prefer to know that such a cleansing device is activated at intervals in response to certain conditions or according to a prescribed cleaning schedule, such as every several minutes. Therefore, there is a need for efficiently cleaning and sanitizing a door handle at prescribed time intervals in response to certain conditions, e.g., according to a predetermined cleaning schedule, opening and closing of the door, passing of an individual in the vicinity of the door knob, turning on/off of the lights, etc. There is also a need to control the door handle sanitizing device to automatically shut down during times when the door is not in use, e.g., overnight, weekends, and any other prescribed time when the door is not being use for an extending period. There is also a need to provide an efficient, compact and effective spray actuator that is part of the cleansing device.

### SUMMARY OF THE DISCLOSURE

According to an embodiment consistent with the present disclosure, a system, device, and method render germ-free door handles in a commercial environment by sanitizing such door handles through the dispensing of a germicide, such as a disinfectant, antibacterial solution, or cleansing agent, onto the door handles at controlled intervals. The controlled intervals are determined by a set of controls which establish a series of settings corresponding to a different amount of time that a valve member is in an operating state.

In an embodiment, a door handle sanitizer system for commercial environments comprises a base and an outer housing, a holder, an electronic valve module, a controller, a set of controls, and a lock. The outer housing is coupled to the base and movable between an open and a closed position. The holder receives and holds a container that stores a germicide and includes a first valve member. The holder is coupled to the base such that when the outer housing is opened, the container can be inserted and removed. The electronic valve module includes a housing that is coupled to the base by engaging locating and support members integrally formed as part of the base. The electronic valve module is positioned relative to the holder such that when the container is in a fully loaded position with respect to the holder, the first valve member is actuated and opened. Discharge of the germicide from the sanitizer onto the door handle to sanitize the door handle is determined by an operating state of a second valve member of the electronic valve module that is located downstream of the first valve member.

The controller has a processor. The controller is operatively connected to a power source and to the electronic valve module for switching the operating state between a first operating state in which there is discharge of the germicide from the sanitizer through the second valve member and a second operating state in which there is no discharge of the germicide. The set of controls is configured to influence the configuration of the electronic valve module by providing input to the controller. The set of controls is supported within an interior of the door handle sanitizer system such that no controls are accessible to a user when the outer housing is in the closed position. Also, the set of controls includes a reset control. The controller is configured by code executing in the processor thereof to respond to the reset control by resetting the electronic valve module to a factory setting in response to a first press of less than one second. Also, the controller responds to the reset control by cycling to a first setting among a series of settings in response to a long press of five seconds. Each setting in the series of settings establishes a different amount of time that the second valve member will be in the first operating state. In addition, the lock is configured to secure the outer housing to the base in the closed position.

A digital display can be mounted so as to be exteriorly visible both when the outer housing is in the open and closed position. The digital display provides feedback as the series of settings cycles changes due to the long press of the reset control. Within a prescribed period of time after the long press, a further press of the reset control causes the processor to cycle to a next setting among the series of settings. With each further press of the reset control, the digital display provides an indication of a current location within the series of settings as the processor responds to the reset control. The series of settings returns to a first setting after the last setting in the cycle has been reached. Also, the further press of the reset control can be a short press of less than one second. In addition, an override button can be mounted exteriorly to the housing. The override button is connected to the controller. The override button provides a signal that momentarily places the second valve member in the first operating state so as to discharge the germicide from the sanitizer for that momentary time. Alternatively, the set of controls includes a variable spray dosage control, such that the controller is configured by code executing in the processor thereof to respond to the variable spray dosage control by selecting an output dose of the second valve member from among a plurality of selectable doses of the germicide when the second valve member is in the first operating state. The plurality of selectable doses can include four different doses of the germicide.

In another embodiment, a method for sanitizing a door handle comprises storing a germicide in a holder and receiving an input from a set of controls to a controller having a processor. The method then actuates, by the controller in response to the received input, an electronic valve module such that a valve member attached to the holder is switched between a first operating state in which there is a discharge of the germicide, and a second operating state in which there is no discharge of germicide. The method further discharges, during the first operating state, the germicide from the holder through the valve member and onto the door handle, thereby sanitizing the door handle. The set of controls includes a reset control and wherein the controller is configured by code executing in the processor thereof to respond to the reset control by resetting the electronic valve module to a factory setting in response to a first press of less than one second, and cycling to a first setting among a series of settings in response to a long press of five seconds, wherein each setting in the series of settings establishes a different amount of time that the valve member will be in the first operating state.

The method also can provide a digital display mounted so as to be exteriorly visible both when the outer housing is in the open and closed position, the digital display providing feedback as the series of settings cycles changes due to the long press of the reset button. Within a prescribed period of time after the long press, a further press of the reset control causes the processor to cycle to a next setting among the series of settings. With each further press of the reset control, the digital display provides an indication of a current location within the series of settings as the processor responds to the reset control. The series of settings can return to a first setting after the last setting in the cycle has been reached. The further press of the reset control can be a short press of less than one second. An override button can be mounted exteriorly to the housing, such that the override button is connected to the controller and provides a signal that momentarily places the second valve member in the first operating state so as to discharge the germicide from the sanitizer for that momentary time.

Alternatively, the set of controls can include a variable spray dosage control. The controller can be configured by code executing in the processor thereof to respond to the variable spray dosage control by selecting an output dose of the valve member from among a plurality of selectable doses of the germicide when the valve member is in the first operating state. The plurality of selectable doses can include four different doses of the germicide.

In a further embodiment, a device comprises a base, an outer housing, a holder, an electronic valve module, a controller, a set of controls, and a lock. The outer housing is coupled to the base and is movable between an open and a closed position. The base and the outer housing retain a self-contained power source. The holder that receives and holds a container that stores a substance and includes a first valve member, with the holder being coupled to the base such that when the outer housing is opened, the container can be inserted and removed. The electronic valve module that includes a housing that is coupled to the base by engaging locating and support members integrally formed as part of the base. The module is positioned relative to the holder such that when the container is in a fully loaded position with respect to the holder, the first valve member is actuated and opened. Discharge of the substance from the device onto a door handle is determined by an operating state of a second valve member of the electronic valve module that is located downstream of the first valve member. The controller has a processor, with the controller being operatively connected to the self-contained power source and to the electronic valve module for switching the operating state between a first operating state in which there is discharge of the substance through the second valve member and a second operating state in which there is no discharge of the substance.

The set of controls is configured to influence the configuration of the electronic valve module by providing input to the controller. The set of controls is supported within an interior of the device such that no controls are accessible to a user when the outer housing is in the closed position. The set of controls includes a reset control and controller is configured by code executing in the processor thereof to respond to the reset control by resetting the electronic valve module to a factory setting in response to a first press of less than one second, and cycling to a first setting among a series of settings in response to a long press of five seconds, wherein each setting in the series of settings establishes a different amount of time that the second valve member will be in the first operating state.

The set of controls also includes a variable spray dosage control and the controller is configured by code executing in the processor thereof to respond to the variable spray dosage control by selecting an output dose of the second valve member from among a plurality of selectable doses of the substance when the second valve member is in the first operating state. The plurality of selectable doses can include four different doses of the substance. The lock is configured to secure the outer housing to the base in the closed position.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

The present invention is illustrated by way of example and not by way of limitation in the figures of the accompanying drawings in which like references indicate similar elements.

FIG. 1 is a front elevation view of a door handle sanitizer according to a first embodiment of the present invention;

FIG. 2 is a side perspective view of the door handle sanitizer of FIG. 1 with a front cover thereof removed;

FIG. 3 is an exploded perspective view of the various parts of the sanitizer of FIG. 1;

FIG. 4 is a perspective view of a cartridge or canister holding a fluid to be dispensed by the sanitizer of FIG. 1;

FIG. 5 is a front elevation view of a door handle sanitizer according to a second embodiment of the present invention;

FIG. 6 is a front perspective view of the door handle sanitizer of FIG. 5 in an open position;

FIG. 7 is an exploded perspective view of the various parts of the sanitizer of FIG. 5; and

FIG. 8 is a flowchart of a method of operation of the sanitizer of FIGS. 1 and 5.

It is noted that the drawings are illustrative and are not necessarily to scale.

### DETAILED DESCRIPTION OF CERTAIN EMBODIMENTS OF THE DISCLOSURE

Example embodiments consistent with the teachings included in the present disclosure are directed to a system, device, and method which render germ-free door handles in a commercial environment by sanitizing such door handles through the dispensing of a germicide, such as a disinfectant, antibacterial solution, or cleansing agent, onto the door handles at controlled intervals. The controlled intervals are determined by a set of controls which establish a series of settings corresponding to a different amount of time that a valve member is in an operating state.

The general design and function of one spray dispenser for a door are disclosed in U.S. Pat. No. 7,320,418, which is hereby incorporated by reference in its entirety.

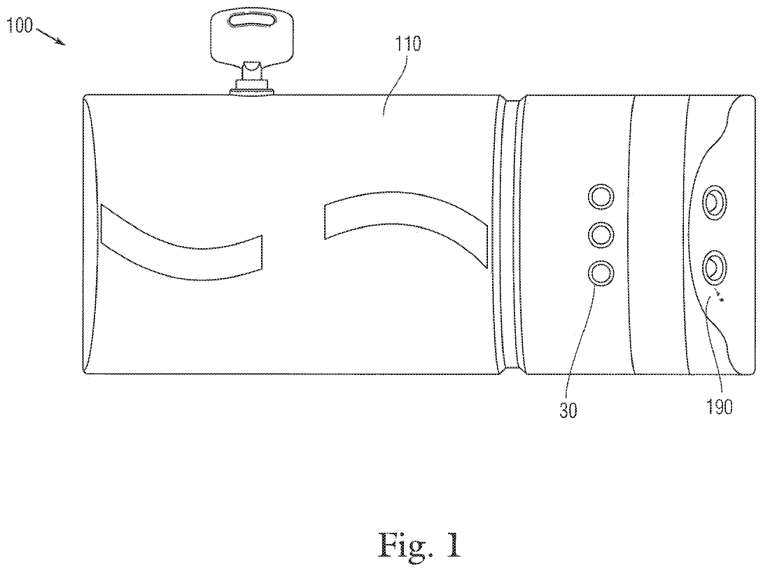

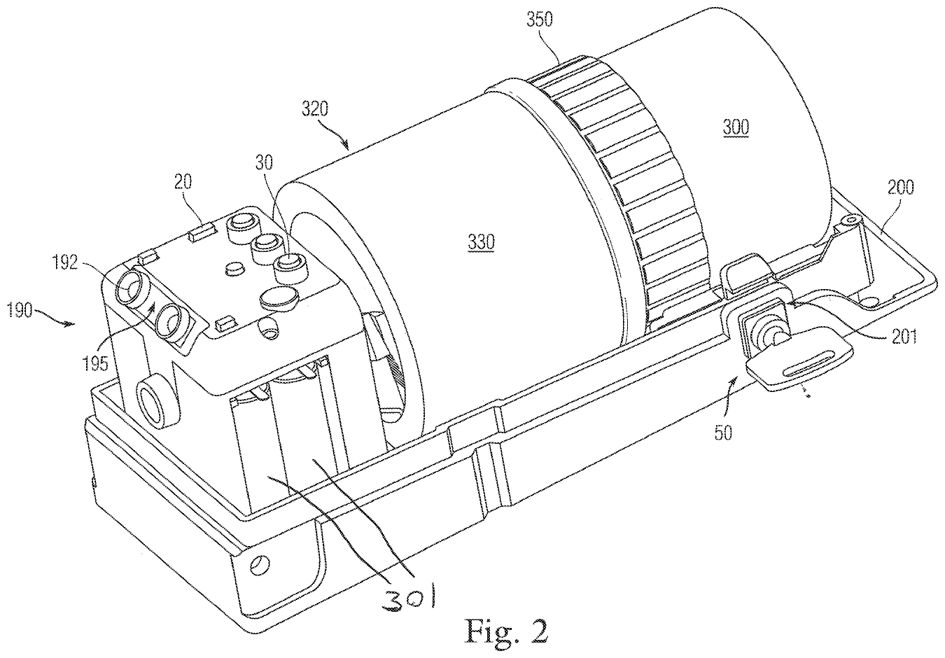

In accordance with the present invention and as shown in FIGS. 1-4, a device can be configured as a spray dispenser 100 in accordance with a first embodiment is shown. The spray dispenser 100 is operable to spray any known substance, such as a germicide 10 therefrom, to coat, and thereby sanitize an outwardly extending door handle situated in an environment, such as a commercial environment. The spray dispenser 100 can be an aerosol air freshener, which has a self-contained power source including batteries 301, shown in FIG. 2.

Referring to FIGS. 1-4, the spray dispenser 100 includes an outer housing or cover 110, a base 200, user accessible control switches 20 and visible indicator lights 30 for providing control and status information of the dispenser 100. The spray dispenser 100 is mounted to the door above the door handler to permit the sprayed germicide to travel down and coat the handle.

The outer housing 110 can be coupled to the base 200 in any number of different ways that allow the outer housing 110 to be opened relative to the base 200. For example, the outer housing 110 can be pivotally or hingedly coupled to the base and in one embodiment, the outer housing 110 is hingedly connected to the base 200 via prongs a top end thereof. In a closed position, the outer housing 110 completely conceals the internal components of the dispenser 100, while in the opened position, the outer housing 110 provides access to a germicide source 300 as well as other internal components contained within the dispenser 100, such as a power source (e.g., one or more batteries 301 shown in FIG. 2). The power source can be self-contained, such as the one or more batteries 301. The batteries 301 can be rechargeable.

In addition, the outer housing 110 can be lockingly coupled to the base 200 using a lock mechanism 50. The lock mechanism 50 can be any number of different lock mechanisms that allow the outer housing 110 to be securely attached to the base 200 when the lock mechanism 50 is placed in a locked position. For example and as shown, the lock mechanism 50 can be a key based latch mechanism that includes an assembly that is inserted into an opening 201 that is formed in the base 200. The assembly includes a key 52 and a base plate 54 that includes a section 55 that is inserted into and is assessable through the opening 201. The base plate 54 includes a first part 57 and a second part 59 that includes the section 55. A hole 54 formed in the section 55 receives a lock part 63 that receives the key 52. The lock part 63 can include one or more tabs extending radially outward such that when the lock part 63 rotates in the hole 54 due to rotation of the key 52, the position of the tabs changes to effectuate unlocking and locking of the outer housing 110. The first part 57 is coupled to a side wall of the base 200 and includes a slot 61 formed along one edge thereof. The second part 59 includes a prong 63 that is sized so that it can be received in the slot 61. To lock and unlock the outer housing 110 from the base 200, the user simply inserts the key 52 through the opening 201 and into the hole 54 and then rotates the key 52 to cause the inner workings of the lock mechanism 50 to move and disengage from the outer housing 110 as in the case of unlocking the outer housing 110 from the base 200. To lock the outer housing 110 to the base 200, the outer housing 110 is placed back on the base 200 and the process is reversed resulting in the engagement of the outer housing 110 to the base 200.

In this embodiment, the outer housing 110 does not have to be hingedly coupled to the base 200 but instead the outer housing 110 can be a separate part that is removable (detachable) from the base 200 when the lock mechanism 50 is in an unlocked position. Alternatively, the outer housing 110 can be hingedly connected along one side so that the outer housing 110 opens and closes like a traditional door as opposed to opening and closing along the top (up and down manner).

The outer housing 110 of the spray dispenser 100 includes a top wall 112, a bottom wall 120 and, for the purpose of presenting a pleasant appearance, a curved front wall 130. The outer housing 110 has a pair of side edges 111 that extend between the top wall 112 and the bottom wall 120. The side edge 111 is not a straight edge but instead is irregular in that it includes a notch (U-shaped) 113 that aligns with the lock mechanism 50 when the outer housing 110 is mated to the base 200. The outer housing 110 can also include a protrusion 115 that mates with a grooved section of the base 200 to further locate and situate the outer housing 110 relative to the base 200.

The outer housing 110 is thus a hollow member that receives a number of the working components of the spray dispenser when the outer housing 110 is in the closed position.

The base 200 is also a generally hollow member that has a rear wall or surface 202, a bottom wall 204 and a pair of side walls 206. The top of the base 200 is substantially open; however, there is a small top wall or surface 208. The bottom wall 204 extends across between the side walls 206 and is joined to the rear wall 202. A top edge 209 of the rear wall 202 extends above the top wall 208. One side wall 206 includes the opening 201 and each of the side walls 206 has a construction that is complementary to the side edges 112 of the outer housing 110. For example, an outer surface of the side wall 206 can include a contoured, recessed section 210 that receives the side edge 112 of the outer housing 110. The recessed section 210 is defined by an edge 212 such that in the closed position, the edges 112, 212 abut one another for a clean appearance. The key hole area formed as part of the side wall 206 is received into the notch 113 of the side edge 112.

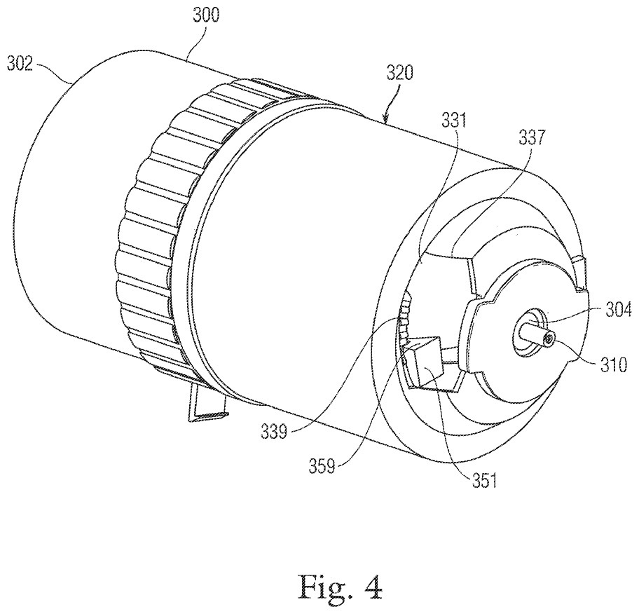

The spray dispenser 100 has a number of working components that permit the germicide to be selectively and controllably discharged from the dispenser 100 as described below. The germicide source 300, preferably in the form of an aerosol can, is removably mounted within spray dispenser 100. In particular, a germicide source 300 can be in the form of an elongated, round aerosol cartridge (can) 300 as shown in FIG. 4. The cartridge 300 has a first end 302 and an opposite second end 304. A valve member 310 is located at the second end 304 and is designed so that when the valve member 310 is actuated (pressed), the valve opens and stored germicide is released.

The dispenser 100 also includes a cartridge holder assembly 320 for holding the cartridge 300 (germicide source) in place within the interior of the dispenser 100. The assembly 320 includes an outer holder or sleeve member 330 and an inner holder or sleeve member 350. The inner sleeve member 350 is a hollow part that has an open top 352 and a partially closed bottom 354 and is sized and shaped to receive at least the second end 304 of the cartridge 300. As a result, the inner sleeve member 350 is a cup shaped member that includes a circular side wall. The cartridge 300 can interface with the inner sleeve member 350 in any number of different manners including a frictional fit. For example, the inner sleeve member 350 can fit over the shoulder of the cartridge 300.

In particular, an opening 353 at the bottom 354 is sized to receive at least the valve assembly 310 of the cartridge 300 such that the valve assembly 310 extends below the bottom 354. At or near the top 352, a plurality of ribs can be circumferentially arranged therearound. The inner sleeve member 350 can be formed a suitable plastic using conventional techniques, such as a molding process. At or near the bottom 354, the inner sleeve member 350 includes one or more locking tabs 351 best shown in FIG. 4. The locking tab 351 includes a locking surface 359 which engages a complementary portion of the outer sleeve member 320 as described below.

Similarly, the outer sleeve member 330 is a hollow part that has an open top 332 and a partially closed bottom 334 and is shaped and sized to receive both the inner sleeve member 350 and the cartridge 300 that is contained therein. As a result, the sleeve member 330 is a cup-shaped member that includes a circular shaped side wall 336. The bottom 334 includes an opening 335 that permits the valve member 310 of the cartridge 300 to be accessible and engageable. In other words, the valve member 310 passes through the opening 335. The outer sleeve member 330 can be coupled to the base 200 using conventional means, such as a mechanical fit, including use of prongs or the like 313 that snap fit into receiving portions of the base 200.

As previously mentioned, the inner and outer sleeve members 350, 330 selectively engage one another for locking the canister/cartridge in place within the holder assembly. For example and as shown in the drawings, the outer sleeve member 330 can include one or more (e.g. a pair) of notches or openings 337 formed in the bottom 334 of the outer sleeve member 330. In the illustrated embodiment, there are a pair of notches 337 formed opposite one another. Within the opening 337, the outer sleeve member 330 has a locking mechanism for selectively yet removable locking the inner sleeve member 350 to the outer sleeve member 330. For example, the locking mechanism can be in the form of a locking rail or edge 339 that is formed along an outer edge of the opening 337. The locking rail 339 includes a series of teeth that are formed along a length of the rail 339. There is a space 331 formed adjacent the locking rail 339, with the space 331 being free of any teeth.

The manner of locking the inner sleeve member 350 to the outer sleeve member 330 is now described. The space 331 is sized and constructed to receive the locking tab 351 that is fixedly formed as part of the inner sleeve member 350 at the bottom 354 thereof. When the inner sleeve member 350 is first inserted into the outer sleeve member 330, the locking tab 351 is aligned with the space 331. When received within the space 331, the locking tab 351 and in particular, the locking surface 359 is located adjacent the teeth of the locking rail 339. The teeth and locking tab are complementary to one another and engage one another in a ratcheting manner in that as the inner sleeve member 350 is rotated (or the cartridge 300 is rotated), the locking surface 359 of the locking tab 351 begins to ride along the locking rail 339 and engage the teeth thereof, thereby securely locking the inner sleeve member 350 in place relative to the outer sleeve member 330. The locking tab 351 thus can function as a pawl of a ratcheting mechanism as it engages the teeth of the locking rail 339. It will therefore be appreciated that the cartridge 300 is securely locked in place in an engaged state by simply inserting the inner sleeve member 350 to which the cartridge 300 is mated into the outer sleeve member 330 in a properly aligned manner and then rotating the inner members relative to the outer sleeve member 330 to cause a locking therebetween. To remove the cartridge 300 as when it is empty, the locking tab 351 is disengaged from the locking rail 339 and the inner sleeve member 350 is rotated in an opposite direction until the locking tab 351 aligns with the space 331 to permit removal of the inner sleeve member 350 and cartridge 300. Other techniques can be used to remove the cartridge 300.

This locking results in the cartridge 300 being properly loaded and the valve assembly 310 is in the proper orientation.

In one embodiment, the cartridge 300 is locked in place by first inserting it into the inner sleeve member 350 and then inserting the inner sleeve member 350 into the outer sleeve member 330 and pushing the cartridge 300 down and then rotating the inner sleeve member 350 relative to the outer sleeve member 330 to cause a locking therebetween. This type of action is similar to the opening and locking of a childproof pill bottle where a force is applied to the cap and the cap is then rotated to cause locking.

The dispenser 100 also includes a spray actuator 400 that controls the regulation and spraying of the germicide. In accordance with the present invention, the actuator 400 is in the form of an electronic valve module 500 that includes a controllable valve, a processor (a printed circuit board (PCB)) 520, a power source (batteries 301) and can include the user accessible control switches 20 and the visible indicator lights 30. In the illustrated embodiment, the actuator 400 has a partially hollow body or housing 410 that is generally square or rectangular shaped and includes a rear wall 412, a partially open front wall 414 and opposing side walls 416. An outer surface of the side walls 416 stores the power source 301 (batteries) and in particular, the outer surface of each of the side walls 416 can include a compartment 421 that receives and includes the power source 301 which is in the form of one or more batteries. In the illustrated embodiment, each side wall 416 includes one compartment 421 that each stores a pair of batteries 301. The actuator 400 has a hollow interior compartment or space 401 that has a top portion that receives the partially closed bottom 334 of the outer sleeve member 330 and allows the germicide to be dispensed as described below.

The actuator 400 also includes the electronic valve module 500 that includes the controllable valve and the processor (circuit board) 520. For example, electronic valve module 500 can be a solenoid and therefore, the valve is a solenoid valve. As is known, the valve is an electrochemical valve for use with liquid or gas controlled by running or stopping an electrical current through a solenoid, which is a coil of wire, thus changing the state of the valve. The electronic valve module 500 is designed to be received and contained within the actuator housing 410 within the hollow interior space 401. The electronic valve module 500 has an inlet 540 that receives the liquid or gas, in this case, the discharged germicide, and an opposite outlet 550 with the solenoid valve being formed between the inlet 540 and the outlet 550. Accordingly, when the valve position is in the open position, the discharged germicide can flow into, through and out of the outlet 550 and conversely, when the valve is closed, the discharged germicide is prevented from flowing into the outlet 550.

The actuator 400 also includes a flow director 560 that includes a housing that receives the electronic valve module 500 and includes an outlet 562 (e.g., tubular shaped conduit) that receives the outlet 550 of the module 500 in such a way that the plume of the discharged germicide is not impeded with and more specifically, the outlet 562 and the outlet 550 both have round cavities or spaces that allow for the natural spray pattern of the germicide has it is discharged from the cartridge 300 through the valve of the module 500.

The processor (circuit board) 520 can be coupled to the body 410 and can extend across the open front wall 414. Electrical connection is made between the electronic valve module (solenoid) 500 and the power source 301 (batteries), thereby powering the electronic valve module 500 and selectively permitting the solenoid valve to be opened and closed depending upon the operating state of the dispenser 100. The user accessible control switches 20 and the visible indicator lights 30 are also operatively connected to the processor 520 to permit control over the dispenser and allow the user to place the dispenser 100 in any number of different operating states as described below.

A control panel cover 580 can be provided for insertion over the user accessible control switches 20 and the visible indicator lights 30. The cover 580 has openings formed therein to permit the control switches 20 and the indicator lights 30 to be accessible and/or visible. The cover 580 is coupled to the actuator housing 410 using conventional means, e.g., snap-fit means. As shown, the body 410 can include a pair of posts 401 that are received into complementary openings 582 formed in the cover 580.

In yet another aspect of the present invention, the dispenser 100 has a sensing mechanism 190 incorporated therein to detect the presence of an unintended object underneath or in close proximity to the dispenser 100. When an unintended object is located too close to the dispenser 100 (within the sensing zone), the actuator 400 is not actuated while the unintended object remains within the sensing zone. For example, if a small child is standing underneath or is in close proximity to the dispenser 100, the sensing mechanism will detect such presence and if this detection event overlaps with a time when the dispenser is programmed to be actuated, the dispenser 100 will not spray germicide from the cartridge 300. Instead, the control program can be designed so that if the dispenser 100 is not actuated at the programmed time due to activation of the sensing mechanism, the program will wait a predetermined (programmed) time period before attempting again to actuate the actuator assembly and spray germicide. This process can continue until a successful actuation occurs and the germicide is sprayed.

The sensing mechanism 190 illustrated in the figures includes a pair of sensors 192 that are located at or near the bottom of the dispenser 100. In the illustrated embodiment, the sensing mechanism 190 is located proximate the other control features and PCB 520 of the dispenser 100. In particular and as shown in FIG. 2, the cover 580 can include openings 584 that accommodate the sensors 192. In addition to the sensors 192 themselves, the sensing mechanism 190 also includes a sensor cover 195 and a sensor base 197 that properly located and positions the sensors 192. The sensor base 197 includes a pair of slots or retaining structures 199 that receive and hold the sensors 192 at the proper angle. The sensors 192 can be set at 45 degree angles (relative to a vertical axis of the dispenser) and can be set at different angles and in different directions from one another resulting in an expanded scope of sensing coverage. The sensors 192 are operatively connected to the electronics (PCB and controller) of the dispenser 100 so that sensor signals are sent to the controller and under select conditions, such as when an unintended object is sensed by sensors 192, the controller, having a processor executing code which configures the processor for particular operations described herein, controls operation of the actuator 400 so that the unintended object that is located close to the dispenser 100 is not sprayed with germicide. Since there are two sensors 192, the sensor cover 195 includes two openings to accommodate the sensors 192. The sensor cover 195 can be snap-fittingly engaged to the cover 580 to allow access to the sensors 192 in the case that access is needed. The main cover 130 also includes openings 589 that allow operation of the sensors 192 since the sensors 192 are axially aligned with the openings 589.

Any number of different sensors 192 can be used in the dispenser 100. For example, the sensors 192 can be in the form of motion sensors or can be sensors that detect an obstruction in a path thereof.

In one embodiment, the sensing mechanism 190 is configured so that it is capable of sensing an object that is located within one and half feet from the floor to the dispenser 100. This is sufficient to detect a small child or animal, etc.

The actuator 400 can be constructed to be removably mated and securely coupled to the base 200. For example, the base 200 can include molded structures, e.g., ribs, etc., that guide and allow for the insertion of the actuator 400 into the base 200. Snap-fit type arrangements or fasteners can be used to couple the actuator 400 to the base 200.

As shown in FIG. 2, in the fully assembled condition, the electronic valve module 500 and most the actuator 400 are disposed below the outer sleeve member 330 and the inserted cartridge 300. It will be appreciated that the cartridge holder and the actuator 400 are complementary to one another and are positioned relative to one another such that when the cartridge 300 is disposed into the inner sleeve member 350 and is locked in place relative to the outer sleeve member 330, the valve member 310 of the cartridge 300 is inserted into the inlet 540 of the module 500 and this insertion causes the cartridge valve member 310 to be opened, thereby causing the germicide to be discharged from the cartridge 300 into the inlet 540. In other words, when the cartridge 300 is locked in place and the cartridge 300 is in the fully loaded position of FIG. 2, the valve member 310 thereof remains in an open position, thereby allowing the germicide to be discharged from the cartridge 300 into the electronic valve module 500. In other words, the loading of the cartridge 300 causes the valve assembly 310 to be moved into the open position. Whether or not the germicide is discharged from the dispenser 100 depends on the position of the solenoid valve. When the solenoid valve is opened, the germicide is discharged from the electronic valve module 500 by passing through the outlet 562 and through the flow director 560. The outlet 562 of the flow director 560 is aligned with the opening formed in the bottom wall 120 of the outer housing 110 and therefore, the spray freely emanates from the dispenser 100.

The control switches 20 and the indicator lights 30 can be similar or identical to those described in the previously incorporated '418 patent. In one embodiment, one control switch 20 is an on/off switch, one is a reset button, and there can be buttons to set the frequency of when the germicide is sprayed. For example, the one control switch 20 is a switch to set how often the germicide is sprayed and in particular, this switch 20 can be moved into a number of different time interval settings, such as 15 minutes, 30 minutes, and 60 minutes. To program the dispenser 100 to spray every 30 minutes, the control switch 20 is set to the 30 minute setting. The control switches are easily accessible once the front cover 130 is lifted or otherwise separated from the base 200. The indicator lights 30 can include a power on light, a light sensor indicator and a refill indicator that indicates replacement of the cartridge 300 is in order.

In accordance with a salient aspect of the disclosure, multiple operating modes are enabled without an increase in the number of controls (e.g., buttons) provided on the dispenser 100. In particular, the controller can be configured by code executing in the processor thereof to respond to a pressing of the reset button by resetting the electronic valve module 500 to a factory setting in response to a first press of less than one second. Also, the controller can respond to the pressing of the reset button by cycling to a first setting among a series of settings in response to a long press of five seconds. Each setting in the series of settings can establish a different amount of time that the controllable valve will be in the first operating state.

The indicator lights 30 provide a digital display. The indicator lights 30 can be mounted so as to be exteriorly visible both when the outer housing is in the open and closed position. The indicator lights 30 provide feedback as the series of settings cycles changes due to the long press of the reset button. Within a prescribed period of time after the long press, a further press of the reset button causes the processor to cycle to a next setting among the series of settings. With each further press of the reset button, the indicator lights 30 provide an indication of a current location within the series of settings as the processor responds to the reset button. The series of settings returns to a first setting after the last setting in the cycle has been reached. Also, the further press of the reset button can be a short press of less than one second. In an alternative embodiment, a display screen (not shown) can be provided in lieu of or in addition to the indicator lights for displaying information to a user, including changes to the operating mode using words or characters presented on the display screen (e.g., the current selected time setting in the cycle).

In addition, the control switches 20 can include an override button mounted exteriorly to the housing. The override button is connected to the controller. The override button provides a signal that momentarily places the controllable member in the first operating state so as to discharge the germicide from the dispenser 100 for that momentary time. In this way, a person approaching a door can activate the dispenser 100 and cause it to dispense a germicide at that moment.

The control switches 20 can further include a variable spray dosage control, such that the controller is configured by code executing in the processor thereof to respond to the variable spray dosage control by selecting an output dose of the controllable valve from among a plurality of selectable doses of the germicide when the controllable valve is in the first operating state. The plurality of selectable doses can include four different doses of the germicide, for example. The dispenser 100 can be an aerosol air freshener. A user of the dispenser 100 can use the variable spray dosage control to determine how much of the substance therein which the user desires to be sprayed. The dosage can be determined according to the size of the space in which the dispenser 100 is used. The dosage can also be determined by the amount of malodor in the space.

It will therefore be appreciated that in the embodiment of FIGS. 1-4, the loading of the cartridge assembly into the actuator assembly 400 results in the valve assembly 310 being permanently depressed and therefore, the valve assembly 310 itself does not have to be contacted or manipulated to cause a spraying of the germicide.

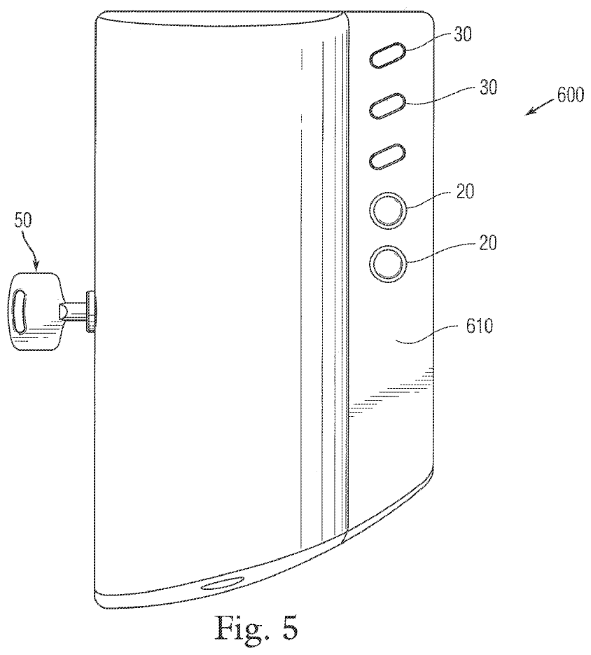

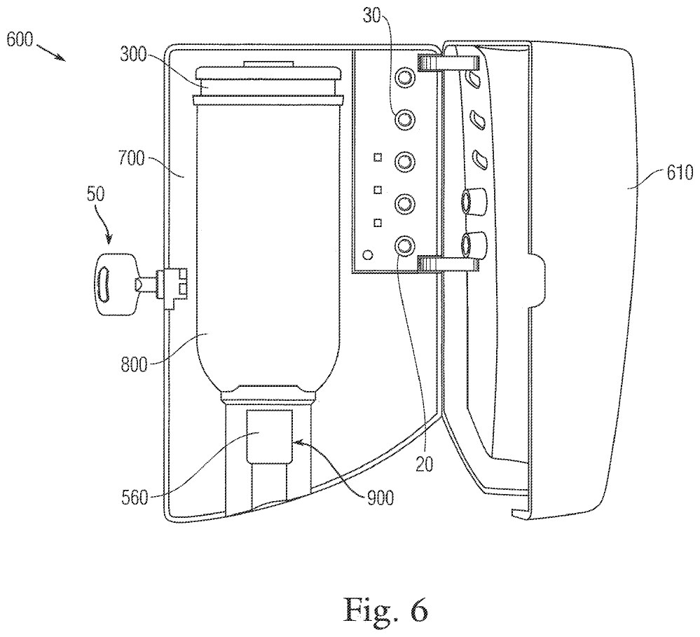

Now referring to FIGS. 5-7 in which a spray dispenser 600 in accordance with a second embodiment is shown. Dispenser 600 is similar to dispenser 100 and therefore, like elements will not be described in great detail. In particular, the layout of the controls 20 and visual indicators 30 is different in the dispenser 600 and in particular, the controls 20 and indicators 30 are aligned in a vertical row as opposed to being arranged in rows. It will be appreciated that the operation and performance of the dispenser 600 is substantially the same or identical to the dispenser 100 with the main difference being the aesthetic look and design of each dispenser.

The dispenser 600 includes a number of the same or similar working components as the dispenser 100 including but not limited to an outer housing 610 and a base 700, lock mechanism 50, cartridge 300 and a cartridge holder assembly 800 and a dispenser actuator 900.

In this embodiment, the base 700 includes a number of integrally formed features (structures) that permit the other components of the dispenser to be secured thereto in a fixed location. For example, the base 700 can include an integral compartment 710 that stores the power source 301 (batteries). The compartment 710 is closed off with a door 712.

The cartridge holder assembly 800 is similar to the previously described holder assembly. In this embodiment, the holder assembly 800 is in the form of two parts, namely, a first upper part 810 and a second bottom part 830. The first upper part 810 is a cup shaped member that has an open top end 812 and a partially closed bottom end 814. A cylindrical side wall 816 provides the cup-shaped form of the first upper part 810.

The first upper part 810 includes a plurality of flexible fingers 820 that are arranged circumferentially about the bottom 814. The bottom 814 of the first upper part 810 has a series of notches or slots 815 that are circumferentially spaced about the bottom 814. Each finger 820 is axially aligned with the notch 815. The fingers 820 can serve to lock and engage the cartridge 300 in place within the first upper part 810.

The first upper part 810 also includes a part 811 that can be used to couple the first upper part 810 to another part, such as a structure that is part of or coupled to the base 700. For example, a holder 840 can be provided for retaining the first upper part 810 in place, with the holder 840 being mounted to the base 700. The holder 840 has a curved surface 842 that is complementary to the cylindrical side wall 816 of the first upper part 810.

The first upper part 810 is designed to mate with and securely hold the cartridge 300 in place similar to the first embodiment disclosed herein. For example, the cartridge 300 can be located in place within the interior of the first upper part 810 using the flexible fingers 820 or other means. For example, the cartridge 300 sits within the first upper part 810 with the bottom end 314 seated against the bottom of the first upper part 810 and the valve assembly 310 extends through the opening 817.

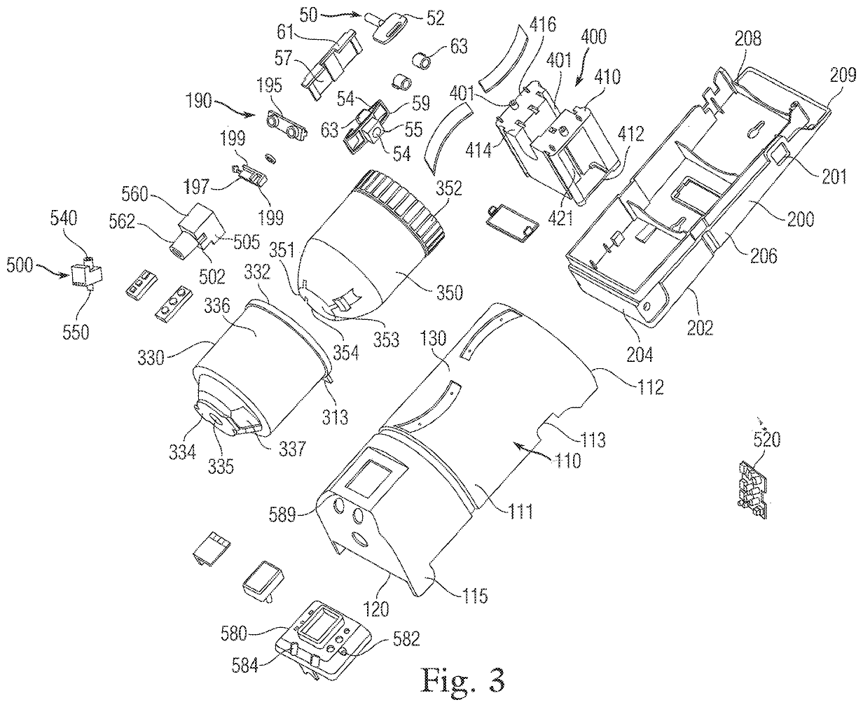

The second bottom part 830 is similarly a cup shaped member that has an open top 832 and a partially closed bottom 834. At the closed bottom end 834, an opening 835 is included and the body of the second bottom part 830 has a side opening or notch 839 formed therein. The second bottom part 830 is designed to securely mate with the first upper part 810 in a manner in which the valve assembly 310 passes through the opening 835 in the bottom part 830. Any number of different techniques can be used to mate the first upper part 810 with the second bottom part 830 including mechanical fit, such as a snap-fit. The engagement between the parts 810, 830 can be similar to the engagement shown in FIG. 3.

Similar to the first embodiment, when the cartridge 300 is inserted into the first upper part 810 and the first upper part 810 is mated with the second bottom part 830, the cartridge 300 is in a loaded position.

The dispenser 600 also includes the spray actuator 900 that controls the regulation and spraying of the germicide. In accordance with the present invention, the actuator 900 is in the form of the electronic valve module 500 (see FIG. 3) that includes a controllable valve, the processor (circuit board) 520, the power source 301 and can include the user accessible control switches 20 and the visible indicator lights 30.

As with the first embodiment, the electronic valve module 500 can be a solenoid and therefore, the valve therein is a solenoid valve. The electronic valve module 500 is designed to be coupled directly to the base 700 at a fixed location that is purposely located in relation to the cartridge holder assembly and in particular, the bottom of the bottom part 830. The flow director or valve module housing 560 contains the electronic valve module 500 and has a rear face or surface 502 that includes a coupling member 505 for coupling the module 500 to the base 700. For example, the coupling member 505 can be in the form of a pair of spaced rails that run vertically along and protrude outwardly from the side edges of the rear face 502. The base 700 includes a complementary coupling feature that mates with the coupling member 205 to couple the module securely, yet detachably 500 to the base 700. For example, the base 700 can include a slot 711 that has a pair of side walls 713 that include capturing lips. The side walls 713 extend out perpendicularly from the rear wall of the base 700 and the lips extend inwardly toward one another and are formed at ends of the side walls 713 and are perpendicular to the side walls 713 (parallel to the rear wall of the base 700). Vertical slots are thus formed between the lips and the rear wall and coupling between the module 500 and the base 700 results by inserting the rails 505 of the module housing 560 into the slots defined by the lips and read rear wall of the base. A stop 509 is formed at one end (e.g., bottom end) of the slot 711 and is designed to limit the travel of the module housing 560. In particular, the module 500 is inserted into the top open portion of the slot 711 and then the rails 505 ride within the slots (resulting in a keying action between the two) in a downward direction until the module housing 560 engages the stop 509. The module housing 560 thus rests on the stop 509. As shown, the stop 509 is in the form of a ledge or the like.

As with the previous embodiment, the electronic valve module 500 includes the inlet 540 that receives the liquid or gas, in this case, the discharged germicide, and the opposite outlet 550 with the solenoid valve being formed between the inlet 540 and the outlet 550. The valve module 500 is receive into the hollow flow director/module housing 560 with the outlet 562 (e.g., tubular shaped conduit) receiving the outlet 550 of the module 500 in such a way that the plume of the discharged germicide is not impeded with and more specifically, the outlet 562 and the outlet 550 both have round cavities or spaces that allow for the natural spray pattern of the germicide has it is discharged from the cartridge 300 through the valve of the module 500.

By incorporating the locating and coupling structure directly into the base 700 and by directly coupling the module housing 560 to the base 700, the electronic module 500 can be easily and accurately placed into a desired position relative to the cartridge 300 such that when the cartridge 300 is loaded into the first upper part 810 and the first upper part 810 and second bottom part 830 are mated to form the cartridge holder structure, the valve member 310 of the cartridge 300 engages and seats within the inlet 540 of the electronic module 500. This coupling between the valve member 310 and the inlet 540 of the electronic valve module 500 results in the valve member 310 being moved to the fully opened position, thereby causing germicide to be discharged into the inlet 540. Consequently, once the solenoid valve is opened, the germicide flows into the outlet 550 of the module 500 and is discharged through the outlet of the module housing 560.

Once again as with the first embodiment, once the cartridge 300 is fully loaded into the cartridge holder, the valve assembly 310 is in the actuated on position.

By fixing the cartridge holder to the base 700 and fixing the valve housing 560 to the same base, the interaction between a fully inserted cartridge and the dispenser actuator (in this case, the solenoid valve) can be carefully controlled to provide a system where the user simply has to insert a full cartridge 300 into the cartridge holder and this action results in the accurate seating of the valve member 310 within the electronic valve module 500 and the opening of the valve member 310. Flow of the germicide is thus controlled completely by the position of the solenoid valve which is in response to user inputted (programmed) selections or in response to other events, such as sensing an object, etc. Exemplary operating parameters are outlined in the '418 patent.

The processor (circuit board) 520 can be coupled to the base 700 and can be inserted into another compartment 709 formed in the base 700. Electrical connection is made between the processor 520, the electronic valve module (solenoid) 500 and the power source 301 (batteries), thereby powering the electronic valve module 500 and selectively permit the solenoid valve to be opened and closed depending upon the operating state of the dispenser 100 and based upon the settings selected or programmed by the user. The user accessible control switches 20 and the visible indicator lights 30 are also operatively connected to the processor 520 to permit control over the dispenser and allow the user to place the dispenser 100 in any number of different operating states. A cover 521 with indicia is disposed over the circuit board 520 and identifies the control switches 20 and lights 30.

As with the first embodiment, the controls of the second embodiment of the present invention can include an on/off switch, a reset button, and buttons to set the frequency of when the germicide is sprayed. For example, the one control switch 20 is a switch to set how often the germicide is sprayed and in particular, this switch 20 can be moved into a number of different time interval settings, such as 15 minutes, 30 minutes, and 60 minutes. To program the dispenser 100 to spray every 30 minutes, the control switch 20 is set to the 30 minute setting. The control switches are easily accessible once the front cover 130 is lifted or otherwise separated from the base 200. The indicator lights 30 can include a power on light, a light sensor indicator and a refill indicator that indicates replacement of the cartridge 300 is in order.

In this embodiment, the controls and indicators are located in the upper right corner of the dispenser above the power source (batteries 301).

In yet another aspect of the present invention and similar to the first embodiment, the dispenser 600 has a sensing mechanism 190 incorporated therein to detect the presence of an unintended object underneath or in close proximity to the dispenser 600. When an unintended object is located too close to the dispenser 600 (within the sensing zone), the actuator 500 is not actuated while the unintended object remains within the sensing zone. For example, if a small child is standing underneath or is in close proximity to the dispenser 600, the sensing mechanism will detect such presence and if this detection event overlaps with a time when the dispenser is programmed to be actuated, the dispenser 600 will not spray germicide from the cartridge 300. Instead, the control program can be designed so that if the dispenser 600 is not actuated at the programmed time due to activation of the sensing mechanism, the program will wait a predetermined (programmed) time period before attempting again to actuate the actuator assembly and spray germicide. This process can continue until a successful actuation occurs and the germicide is sprayed.

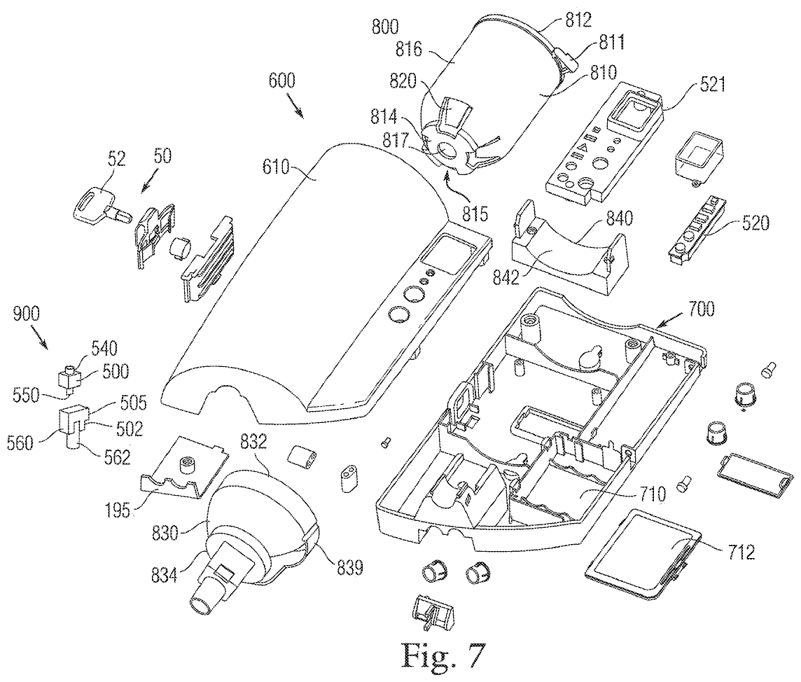

The sensing mechanism 190 illustrated in the figures includes a pair of sensors that are located at or near the bottom of the dispenser 600. In the illustrated embodiment, the sensing mechanism 190 is located proximate the other control features and PCB 520 of the dispenser 600. In particular and as shown in FIG. 7, the cover 610 can include openings that accommodate the sensors 192. In addition to the sensors 192 themselves, the sensing mechanism 190 also includes a sensor cover 195 that properly locates and positions the sensors 192. The sensor cover 195 includes a pair of slots or notches that receive and hold the sensors at the proper angle. The sensors can be set at 45 degree angles and can be set at different angles from one another resulting in an expanded scope of sensing coverage. The sensors are operatively connected to the electronics (PCB and controller) of the dispenser 600 so that sensor signals are sent to the controller and under select conditions, such as when an unintended object is sensed by sensors 192, the controller controls operation of the actuator 500 so that the unintended object that is located close to the dispenser 600 is not sprayed with germicide. Since there are two sensors, the sensor cover 195 includes two openings to accommodate the sensors. The sensor cover 195 can be snap-fittingly engaged to the cover 610 to allow access to the sensors in the case that access is needed.

Any number of different sensors can be used in the dispenser 600. For example, the sensors can be in the form of motion sensors or can be sensors that detect an obstruction in a path thereof. In one embodiment, the sensing mechanism 190 is configured so that it is capable of sensing an object that is located within one and half feet from the floor to the dispenser 600. This is sufficient to detect a small child or animal, etc.

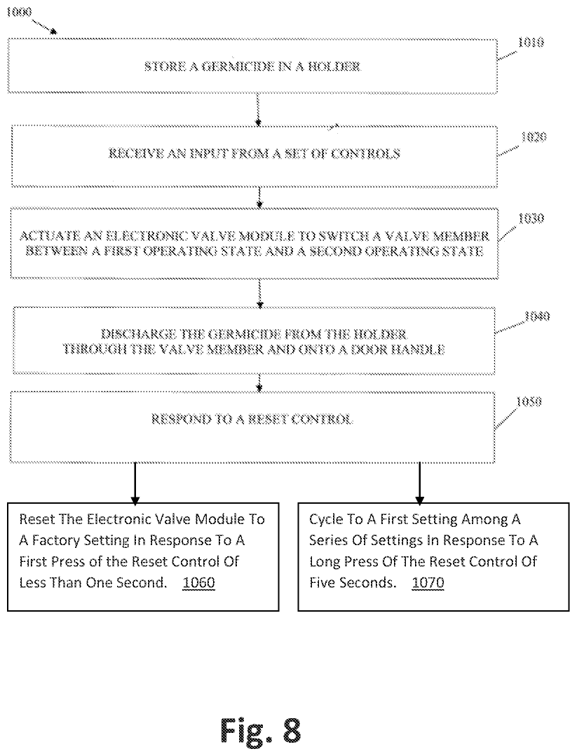

The dispensers 100, 600 in the example embodiments operate according to a method 1000 for sanitizing a door handle, as shown in FIG. 8. The method 1000 comprises storing a germicide in a holder in step 1010 and receiving an input from a set of controls to a controller having a processor in step 1020. In step 1030, the method 1000 then actuates, by the controller in response to the received input, an electronic valve module such that a valve member attached to the holder is switched between a first operating state in which there is a discharge of the germicide, and a second operating state in which there is no discharge of germicide. In step 1040, the method 1000 further discharges, during the first operating state, the germicide from the holder through the valve member and onto the door handle, thereby sanitizing the door handle.

The method 1000 can further include responding to a reset control in step 1050. The controller is configured by code executing in the processor thereof to respond to the reset control by resetting the electronic valve module to a factory setting in step 1060 in response to a first press of less than one second, and cycling to a first setting among a series of settings in step 1070 in response to a long press of five seconds. Each setting in the series of settings establishes a different amount of time that the valve member will be in the first operating state and does so without adding further controls to the dispenser 100.

Portions of the methods described herein can be performed by software or firmware in machine readable form on a tangible (e.g., non-transitory) storage medium. For example, the software or firmware can be in the form of a computer program including computer program code adapted to cause the system to perform various actions described herein when the program is run on a computer or suitable hardware device, and where the computer program can be embodied on a computer readable medium. Examples of tangible storage media include computer storage devices having computer-readable media such as disks, thumb drives, flash memory, and the like, and do not include propagated signals. Propagated signals can be present in a tangible storage media. The software can be suitable for execution on a parallel processor or a serial processor such that various actions described herein can be carried out in any suitable order, or simultaneously.

It is to be further understood that like or similar numerals in the drawings represent like or similar elements through the several figures, and that not all components or steps described and illustrated with reference to the figures are required for all embodiments or arrangements.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation are used herein merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third) is for distinction and not counting. For example, the use of “third” does not imply there is a corresponding “first” or “second.” Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

What is claimed is:

1. A door handle sanitizer system for commercial environments, comprising:
a base and an outer housing coupled to the base and movable between an open and a closed position;
a holder that receives and holds a container that stores a germicide and includes a first valve member, the holder being coupled to the base such that when the outer housing is opened, the container can be inserted and removed;
an electronic valve module that includes a housing that is coupled to the base by engaging locating and support members integrally formed as part of the base, the module being positioned relative to the holder such that when the container is in a fully loaded position with respect to the holder, the first valve member is actuated and opened, whereby discharge of the germicide from the sanitizer onto the door handle to sanitize the door handle is determined by an operating state of a second valve member of the electronic valve module that is located downstream of the first valve member;
a controller having a processor, the controller being operatively connected to a power source and to the electronic valve module for switching the operating state between a first operating state in which there is discharge of the germicide from the sanitizer through the second valve member and a second operating state in which there is no discharge of the germicide; and
a set of controls configured to influence the configuration of the electronic valve module by providing input to the controller,
wherein the set of controls is supported within an interior of the door handle sanitizer system such that no controls are accessible to a user when the outer housing is in the closed position,
wherein the set of controls includes a reset control and wherein the controller is configured by code executing in the processor thereof to respond to the reset control by:
(i) resetting the electronic valve module to a factory setting in response to a first press of less than one second; and
(ii) cycling to a first setting among a series of settings in response to a long press of five seconds, wherein each setting in the series of settings establishes a different amount of time that the second valve member will be in the first operating state; and
a lock configured to secure the outer housing to the base in the closed position.

2. The system of claim 1, further comprising a digital display mounted so as to be exteriorly visible both when the outer housing is in the open and closed position, the digital display providing feedback as the series of settings cycles changes due to the long press of the reset control.

3. The system of claim 2, wherein, within a prescribed period of time after the long press, a further press of the reset control causes the processor to cycle to a next setting among the series of settings.

4. The system of claim 3, wherein with each further press of the reset control, the digital display provides an indication of a current location within the series of settings as the processor responds to the reset control.

5. The system of claim 1, wherein the series of settings returns to a first setting after the last setting in the cycle has been reached.

6. The system of claim 3, wherein the further press of the reset control is a short press of less than one second.

7. The system of claim 1, wherein an override button is mounted exteriorly to the housing, the override button is connected to the controller and provides a signal that momentarily places the second valve member in the first operating state so as to discharge the germicide from the sanitizer for that momentary time.

8. The system of claim 1, wherein the set of controls includes a variable spray dosage control and wherein the controller is configured by code executing in the processor thereof to respond to the variable spray dosage control by selecting an output dose of the second valve member from among a plurality of selectable doses of the germicide when the second valve member is in the first operating state.

9. The system of claim 8, wherein the plurality of selectable doses includes four different doses of the germicide.

10. A method for sanitizing a door handle, comprising:
storing a germicide in a holder;
receiving an input from a set of controls to a controller having a processor;
actuating, by the controller in response to the received input, an electronic valve module such that a valve member attached to the holder is switched between a first operating state in which there is a discharge of the germicide, and a second operating state in which there is no discharge of germicide; and
discharging, during the first operating state, the germicide from the holder through the valve member and onto the door handle, thereby sanitizing the door handle,
wherein the set of controls includes a reset control and wherein the controller is configured by code executing in the processor thereof to respond to the reset control by:
(i) resetting the electronic valve module to a factory setting in response to a first press of less than one second; and
(ii) cycling to a first setting among a series of settings in response to a long press of five seconds, wherein each setting in the series of settings establishes a different amount of time that the valve member will be in the first operating state.

11. The method of claim 10, further comprising providing a digital display mounted so as to be exteriorly visible both when the outer housing is in the open and closed position, the digital display providing feedback as the series of settings cycles changes due to the long press of the reset control.

12. The method of claim 11, wherein, within a prescribed period of time after the long press, a further press of the reset control causes the processor to cycle to a next setting among the series of settings.

13. The method of claim 12, wherein with each further press of the reset control, the digital display provides an indication of a current location within the series of settings as the processor responds to the reset control.

14. The method of claim 10, wherein the series of settings returns to a first setting after the last setting in the cycle has been reached.

15. The method of claim 12, wherein the further press of the reset control is a short press of less than one second.

16. The method of claim 10, wherein an override button is mounted exteriorly to the housing, the override button is connected to the controller and provides a signal that momentarily places the second valve member in the first operating state so as to discharge the germicide from the sanitizer for that momentary time.

17. The method of claim 10, wherein the set of controls includes a variable spray dosage control and wherein the controller is configured by code executing in the processor thereof to respond to the variable spray dosage control by selecting an output dose of the valve member from among a plurality of selectable doses of the germicide when the valve member is in the first operating state.

18. The method of claim 17, wherein the plurality of selectable doses includes four different doses of the germicide.

19. A device, comprising:
a base and an outer housing coupled to the base and movable between an open and a closed position, the base and the outer housing retaining a self-contained power source;
a holder that receives and holds a container that stores a substance and includes a first valve member, the holder being coupled to the base such that when the outer housing is opened, the container can be inserted and removed;
an electronic valve module that includes a housing that is coupled to the base by engaging locating and support members integrally formed as part of the base, the module being positioned relative to the holder such that when the container is in a fully loaded position with respect to the holder, the first valve member is actuated and opened, whereby discharge of the substance from the device onto a door handle is determined by an operating state of a second valve member of the electronic valve module that is located downstream of the first valve member;
a controller having a processor, the controller being operatively connected to the self-contained power source and to the electronic valve module for switching the operating state between a first operating state in which there is discharge of the substance through the second valve member and a second operating state in which there is no discharge of the substance; and
a set of controls configured to influence the configuration of the electronic valve module by providing input to the controller,
wherein the set of controls is supported within an interior of the device such that no controls are accessible to a user when the outer housing is in the closed position,
wherein the set of controls includes a reset control and wherein the controller is configured by code executing in the processor thereof to respond to the reset control by:
(i) resetting the electronic valve module to a factory setting in response to a first press of less than one second; and
(ii) cycling to a first setting among a series of settings in response to a long press of five seconds, wherein each setting in the series of settings establishes a different amount of time that the second valve member will be in the first operating state;
wherein the set of controls includes a variable spray dosage control and wherein the controller is configured by code executing in the processor thereof to respond to the variable spray dosage control by selecting an output dose of the second valve member from among a plurality of selectable doses of the substance when the second valve member is in the first operating state; and
a lock configured to secure the outer housing to the base in the closed position.

20. The device of claim 19, wherein the plurality of selectable doses includes four different doses of the substance.
